SEQUENCE LISTING (5 sequences)

SEQ ID NO: 1 - DNA, 20 bp, Artificial sequence
  Synthetic CpG oligonucleotide TLR9 activating
tccatgacgttcctgacgtt

SEQ ID NO: 2 - DNA, 20 bp, Artificial Sequence
  Synthetic Class A CpG oligonucleotide
gggggacgatcgtcgggggg

SEQ ID NO: 3 - DNA, 24 bp, Artificial Sequence
  Synthetic Class B CpG oligonucleotide
tcgtcgttttgtcgttttgtcgtt

SEQ ID NO: 4 - DNA, 25 bp, Artificial Sequence
  Synthetic Class C CpG oligonucleotide
tcgtcgtcgttcgaacgacgttgat

SEQ ID NO: 5 - DNA, 22 bp, Artificial Sequence
  Synthetic Class B CpG oligonucleotide
tgactgtgaacgttcgagatga

(19) United States
(12) Patent Application Publication
(10) Pub. No.: US 2009/0306190 A1
(43) Pub. Date: Dec. 10, 2009

(54) NEUROPROTECTANTS

(76) Inventors: Mary Stenzel-Poore, Lake Oswego, OR (US); Susan Stevens, Portland, OR (US)

Correspondence Address: KLARQUIST SPARKMAN, LLP, 121 SW SALMON STREET, SUITE 1600, PORTLAND, OR, 97204, US

(21) Appl. No.: 12/066,260

(22) PCT Filed: Sep. 8, 2006
(86) PCT No.: PCT/US06/34797

§ 371 Date: Nov. 24, 2008

Related U.S. Application Data
(60) Provisional application No. 60/715,881, filed on Sep. 9, 2005.

(51) Int. Cl. A61K 31/7088; A61K 31/437; A61P 9/10

(52) U.S. Cl. 514/44 R; 514/293

(57) ABSTRACT

Methods of protecting cells against cytotoxic insults are provided. The methods involve administering a composition including a CpG oligonucleotide to a subject. The methods are applicable to the protection of neural and non-neural cells. For example, methods of protecting a neural cell against excitotoxic brain injury are provided. Methods for preparing medicaments for the prophylactic treatment of excitotoxic injury, ischemia and/or hypoxia are also provided. Also provided are compositions for use in the described methods.

### CROSS-REFERENCE TO RELATED APPLICATIONS

This application claims benefit of the filing date of U.S. Provisional Application No. 60/715,881, filed Sep. 9, 2005, the disclosure of which is incorporated herein in its entirety.

### ACKNOWLEDGMENT OF GOVERNMENT SUPPORT

Aspects of this invention were made with United States government support pursuant to grant no. POI NS 35965 from the National Institute of Neurological Disorders and Stroke (NINDS). The United States government may have certain rights in the invention.

### FIELD

This disclosure relates to the field of neurology. More specifically, the present disclosure relates to the prevention of cellular and organ damage due to excitotoxic injury, ischemia and/or hypoxia by administering an agent that binds to and activates a cellular Toll-like receptor.

### BACKGROUND

The need for preconditioning therapies to reduce the adverse effects of ischemic and other hypoxic conditions is enormous. For example, transient ischemic attacks (TIA's) precede infarction in 25-50% of patients with occlusive cerebral vascular disease, and 50% of patients that undergo coronary artery bypass surgery (CABG) suffer permanent cognitive decline from intraoperative emboli. Perioperative treatment of CABG patients alone (336,000 annually) could reduce stroke incidence and morbidity significantly. Furthermore, individuals who have had a stroke are at high risk of recurrent stroke (25-40% within 5 years).

Decades of research investigating stroke pathogenesis and treatment have revealed robust neuroprotective treatments in the laboratory, however, all have failed to translate into treatments for patients (Plum, J. Am. Med. Assoc., 285:1760-1761, 2001; DeGraba and Pettigrew, Neurol. Clin. 18:475-493, 2000). The failure of a pharmacologic approach to induce neuroprotection in humans may be due to trial design, dose response or time window issues of selected compounds or side effects of study agents. However, all cytoprotective trials over a 25 year period have been negative.

Ischemic tolerance in the brain—in which one or more brief ischemic insults increase resistance to subsequent injurious ischemia—is a powerful adaptive defense that involves an endogenous program of neuroprotection (Nandagopal et al., J. Pharm. Exp. Ther. 297:474-478, 2001; Chen et al., J. Cereb. Blood Flow Metab. 16:566-577, 1996; and reviewed in Dirnagl et al., Trends Neruosci. 26:248-254, 2003). This neuroprotective program sets into motion a complex cascade of signaling events, leading to synthesis of new proteins, that ultimately re-programs the cellular response to subsequent injury. The sequence of events that leads to ischemic tolerance is only partially known (Nandagopal et al., J. Pharm. Exp. Ther. 297:474-478, 2001), although evidence is emerging that diverse stimuli that trigger preconditioning may share a common pathway that confers neuroprotection (Gonzalez-Zulueta et al., Proc. Natl. Acad. Sci. USA 97:436-441, 2000; Kasischke et al., Neurosci. Lett. 214:175-178, 1996; Gidday et al., J. Cereb. Blood Flow Metab. 19:331-340, 1999; Kato et al., Neurosci. Lett. 139:118-121, 1992).

Tolerance to ischemic brain injury can be induced by several distinct preconditioning stimuli including non-injurious ischemia, cortical spreading depression, brief episodes of seizure, exposure to anesthetic inhalants, and low doses of endotoxin (lipopolysaccharide, LPS) (Simon et al., Neurosci. Lett. 163:135-137, 1993; Chen and Simon, Neurology, 48:306-311, 1997; Kitagawa et al., Brain Res. 528:21-24, 1990; Kobayashi et al., J. Cereb. Blood Flow Metab. 15:721-727, 1995; Kapinya et al., Stroke 333:1889-1898, 2002; Towfighi et al., Dev. Brain. Res. 113:83-95, 1999). Although the mechanisms that underlie these processes, and preconditioning in general, are not well understood, they may share a common link that small doses of an otherwise harmful stimulus induce protection against subsequent injurious challenge (Dirnagl et al., Trends Neruosci. 26:248-254, 2003).

LPS, given in small doses, confers profound neuroprotection against subsequent stroke. Certain features of LPS-induced neuroprotection make it an extremely promising target for stroke therapy: 1) systemic delivery of LPS induces robust neuroprotection, thus blood-brain barrier issues are not a concern; 2) neuroprotection occurs rapidly—within one day of administration and perhaps sooner; and 3) neuroprotection lasts at least one week following LPS treatment. However, LPS is poorly tolerated by human and animal subjects. Therefore, alternatives to LPS for preconditioning against stroke and other hypoxic and/or excitotoxic injuries are needed.

### SUMMARY OF THE DISCLOSURE

The present disclosure relates to methods and compositions for protecting cells against cytotoxic insult. The methods disclosed herein are applicable to the protection of neural as well as non-neural cells, and are relevant for the prevention of adverse outcomes due to diverse medical conditions, including epilepsy, traumatic brain injury, in utero hypoxia, ischemic events (including stroke) and Alzheimer's disease, as well as surgical and non-surgical trauma. The methods involve systemically administering a composition (medicament) that elicits a preconditioning effect to a subject. Typically, the composition is administered prior to the excitotoxic and/or hypoxic event, or prior to one or more events in a series of events or during the occurrence of an ongoing or progressive process. Thus, the disclosure relates to methods for the prophylactic treatment of cellular injury and death due to cytotoxic insults, such as excitotoxic, ischemic and/or hypoxic events.

The foregoing and other objects, features, and advantages will become more apparent from the following detailed description, which proceeds with reference to the accompanying figures.

### BRIEF DESCRIPTION OF THE DRAWINGS

FIG. 1 is a bar graphs illustrating neuroprotection following in vitro treatment with an exemplary CpG oligonucleotide. Values are group means+/−SEM; *P<0.05.

FIG. 2 is a bar graph illustrating neuroprotection following in vitro treatment with imiquimod. Values are means+/−SEM.

FIG. 3 is a bar graph illustrating NF-κB induction following in vitro treatment with an exemplary CpG oligonucleotide. 293-hTLR9 cells transfected with an NFκB inducible reporter plasmid (pNifty2-SEAP) were treated with CpG (5 μM). NFκB induction of alkaline phosphatase expression is indicated as hydrolysis of pNpp at 405 nm.

FIG. 4 is a bar graph illustrating neuroprotection in vivo following treatment of mice with an exemplary CpG oligonucleotide. CpG treatment was given 72 hrs prior to induced stroke. Values are group means+/−SEM; *p<0.05, **p<0.001.

FIG. 5 is a bar graph illustrating a time course of preconditioning in vivo with an exemplary CpG oligonucleotide. CpG dose 20 ug/mouse (0.8 mg/kg). Values are group means+/−SEM; **p<0.0001.

FIG. 6 is a bar graph illustrating neuroprotection in vivo following treatment of mice with imiquimod. Values are group means±SEM; (saline n=6, imiquimod n=3); *p<0.05.

### DESCRIPTION OF THE SEQUENCE LISTING

The nucleic and amino acid sequences listed herein and/or in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand.

SEQ ID NO:1 (5′-tccatgacgttcctgacgtt-3′) is an exemplary oligonucleotide that binds to and activates mouse TLR9.

SEQ ID NO:2 (5′-gggggacgatcgtcgggggg-3′) is an exemplary human Class A CpG oligonucleotide.

SEQ ID NO:3 (5′-tcgtcgttttgtcgttttgtcgtt-3′) is an exemplary human Class B CpG oligonucleotide.

SEQ ID NO:4 (5′-tcgtcgtcgttcgaacgacgttgat-3′) is an exemplary human Class C CpG oligonucleotide.

SEQ ID NO:5 (5′-tgactgtgaacgttcgagatga-3′) is an exemplary human Class B CpG oligonucleotide.

### DETAILED DESCRIPTION

The present disclosure concerns methods for protecting cells in vivo, in the context of a living multicellular organism, from the adverse effects of cytotoxic insults, such as excitotoxic, ischemic and/or hypoxic events. More specifically, methods disclosed herein involve preconditioning cells to increase tolerance to subsequent excitotoxic, ischemic and/or hypoxic events. The present disclosure provides novel methods, based on the observation that oligonucleotides including an unmethylated CpG motif are cytoprotective when used in a preconditioning regimen, for protecting cells against excitotoxic injury, ischemia and hypoxia. Administration of CpG oligonucleotides and/or other preconditioning agents (e.g., prior to an excitotoxic, ischemic or hypoxic event) induces cellular and metabolic changes by modifying the genomic response program, which results in resistance to subsequent damage that would otherwise result from excessive electrochemical activity and/or oxygen deprivation.

Thus, one aspect of the disclosure concerns methods of protecting a cell (or population of cells, or a tissue, organ or organism) against cytotoxic insult, including excitotoxic injury, ischemia, hypoxia or a combination of thereof. The methods disclosed herein are applicable to different cell types susceptible to excitotoxic, ischemic and/or hypoxic injury, which are amenable to preconditioning. For example, neural cells (including, e.g., hippocampal neurons and cortical neurons), muscle cells (including cardiac, smooth and striated muscle cells), hepatic cells, renal cells and endothelial cells can be protected against excitotoxic injury, ischemia and/or hypoxia using the methods disclosed herein. Additionally, certain cells of the immune system, including macrophages and microglia are amenable to preconditioning.

The disclosed methods can be utilized to protect cells from cytotoxic insult, for example, arising from excitotoxic, ischemic and/or hypoxic events. That is, the methods are useful for protecting cells from a broad range of events and occurrences that include an excitotoxic, ischemic or hypoxic component, or a combination thereof. Excitotoxic injury results from excessive stimulation of cells (typically neural cells in the CNS) by certain neurotransmitter (e.g., glutamate) receptors. For example, excitotoxic injury can be a result of a condition that causes excessive chemical or electrical activity in the brain or it can be a result of conditions that cause a decrease in inhibitory or regulatory functions of the brain. Excitotoxic injury in the brain is associated with a variety of conditions with disparate etiologies and symptoms, including epilepsy, traumatic brain injury and Alzheimer's disease. Hypoxia in the central nervous system (CNS) can be associated with ischemic events (such as cerebrovascular ischemia, or stroke, myocardial ischemia due to narrowing or blockage of the vessels of the heart, iatrogenic ischemia, due to surgical procedures, and the like). In addition, hypoxia can occur in utero due to conditions such as inadequate placental function (for example, due to abrupio placentae), preeclamptic toxicity, prolapse of the umbilical cord, or complications from anesthetic administration. Ischemic events outside the CNS can also result in injury to tissues and organs, including kidney, liver and muscle. Such injury can be the result of vascular disease or injury, as well as a complication of surgical procedures (e.g., cardiovascular surgery). Additionally, injury by some hypoxic events (such as strokes) involves an excitotoxic component as well as a hypoxic component and are, in some but not all cases related to ischemic events. Thus, it will be appreciated that these terms can be extensively overlapping, but are not necessarily coextensive in every condition that is amenable to preconditioning. For simplicity of reference, in the context of this disclosure, the term “cytotoxic insult” is used to refer to any of these conditions, separately or in any combination. The methods disclosed herein are useful for preventing cellular damage in any (and/or all) of these conditions.

Accordingly, the methods can involve selecting a subject at risk for one or more of an excitotoxic, ischemic or hypoxic event. In the context of the methods described throughout this disclosure, risk is indicated by a variety of medical as well as non-medical indicators, as would be recognized by one of ordinary skill in the art. For example, various cardiovascular signs and symptoms, such as atrial fibrillation, angina pectoris, hypertension, transient ischemic attacks and prior stroke, are all indicators of risk that can be used to select a subject for administration of preconditioning agent according to the methods disclosed herein. Similarly, surgical procedures, especially those specifically involving the cardiovascular system, such as endarterectomy, pulmonary bypass and coronary artery bypass surgeries, are indicators of risk that can be used to select a subject for administration of a preconditioning agent.

In addition, non-medical indicators of risk, for example, pertaining to behaviors or activities that are statistically associated with an increased likelihood of injuries, can include an excitotoxic or hypoxic component. For example, traumatic brain injury (regardless of its cause) frequently involves an excitotoxic (and can also include a hypoxic) component. Thus, participation in activities that increase the risk of traumatic brain injury are indicators that can be used to select a subject for administration of a preconditioning agent (such as a CpG oligonucleotide). Such activities include, for example, motorcycle riding, motor vehicle racing, skiing, contact sports (such as, football, hockey, rugby, soccer, lacrosse, martial arts, boxing and wrestling), and the like. Additionally, impacts or wounds resulting from gunshot or explosives frequently cause traumatic brain injury. Accordingly, activities that are associated with an increased risk of gunshot wounds or injury caused by explosive devices (for example, in combat situations) are indicators of risk that can be used to select a subject for treatment according to the methods disclosed herein.

In certain embodiments, the methods involve systemically administering a composition including an oligonucleotide (or a mixture of oligonucleotides) comprising an unmethylated CpG motif. Typically, a composition including a CpG oligonucleotide is administered to a subject (such as a human subject) at risk for a cytotoxic insult, such as an excitotoxic, ischemic and/or hypoxic event. Commonly, the composition containing the CpG oligonucleotide(s) is a pharmaceutical composition or medicament, formulated for administration to a subject. Such compositions commonly include a pharmaceutical carrier or excipient. Generally, the composition is formulated based on the intended route of administration. Suitable routes of administration include intranasal, oral, transdermal, subcutaneous, intrathecal, intravenous and intraperitoneal routes, and appropriate pharmaceutical carriers for these administration routes are well known in the art. Thus, the use of a CpG oligonucleotide in the preparation of a medicament for the prophylactic treatment of an excitotoxic injury, ischemia or hypoxia (or an increased risk thereof) is a feature of this disclosure.

The composition is typically administered prior to an event or activity associated with (e.g., that increases the risk of) excitotoxic injury, ischemia and/or hypoxia. Generally, at least one dose of the composition is administered at least 10 hours prior to the event or activity, in order to better realize the preconditioning effect of administration. Usually, the composition is administered at least 24 hours before the event or activity. The protective effects of a single administration of a CpG composition can last for greater than one week (e.g., up to about 10 days, or more). Thus, in the case of an isolated event, that is, an event that is not predicted to be a recurring event, such as a surgical operation, the composition is given prior to the commencement of the event, such as about 10 hours, or about 12 hours, or about 24 hours prior to the event or activity, and can be given up to about 1 week prior to the event. Optionally, multiple doses of the composition are administered prior to the commencement of the event (e.g., surgery). For example, two, or three, or more doses can be administered on separate occasions preceding the event. In such cases, a first dose is typically given between 8 and 10 days, at seven days, at six days, at five days, at four days, at three days, at two days, or at 1 day prior to the event. One or more subsequent administrations of the compositions can be made at any subsequent time point, such as at seven days, at six days, at five days, at four days, at three days, at two days, at 24 hours or at 12 hours prior to the event.

In the case of a recurrent event, such as repeated engagement in a contact sport, multiple administrations are given, the ultimate dose (that is, the most recent dose prior to the event) being given prior (such as, at least 10 hours, or up to about 1 week, prior) to the event or activity. Similarly, in the case of an ongoing event, such as in the case of Alzheimer's disease, multiple administrations are given, for example on a predetermined schedule, such as at weekly intervals. The individual treatment regimen can be customized to the particular subject event or activity, such that the protective effects of the preconditioning dose of the CpG oligonucleotide are optimized under the particular circumstances for the particular subject.

Typically, the dose of the composition including the CpG oligonucleotide administered is a preconditioning dose. That is, a dose of the composition is administered that is sufficient to induce cellular changes (for example, in the genomic response) that protect the cell against injury resulting from a subsequent cytotoxic insult, such as an excitotoxic, ischemic or hypoxic event. Methods for detecting such genomic changes are described hereinbelow, e.g., in Example 2. Typically, a preconditioning dose (for example, in a human) includes at least 0.005 mg/kg, such as about 0.01 mg/kg of the oligonucleotide. Usually the dose contains no more than about 0.8 mg/kg of the oligonucleotide. For example, a preconditioning dose can include between 0.01 mg/kg and 0.25 mg/kg of a CpG oligonucleotide, such as between 0.05 mg/kg and 0.2 mg/kg of the CpG oligonucleotide. Certain exemplary doses include about 0.07, about 0.08, about 0.09, about 0.10, about 0.12 or about 0.15 mg/kg of a CpG oligonucleotide.

Following administration of a composition containing the (or multiple different) CpG oligonucleotides, the oligonucleotide(s) binds to and activates a Toll-like receptor 9 (TLR9). Binding of TLR9 by a suitable CpG oligonucleotide ligand results in the activation of intracellular signaling pathways that modify the genetic program in cells expressing the receptor. These modifications in the genomic response include an increase in the production of certain cytoprotective cytokines. For example, binding of a CpG oligonucleotide to TLR9 on the cell surface of certain immune cells, such as B cells, dendritic cells, macrophages and microglial cells induces production of transforming growth factor-beta (TGFβ), tumor necrosis factor-alpha (TNFα) and type I interferons, such as interferon-beta (IFNβ). Thus, representative methods disclosed herein involve administering a CpG oligonucleotide capable of inducing production of one or more cytoprotective cytokines, such as TGFβ, TNFα, and IFNβ.

Numerous CpG oligonucleotides have been described, and are known to bind to TLR9 and induce cellular signaling pathways. Any of these oligonucleotides can be used in the context of composition for preconditioning a cell against excitotoxic or hypoxic injury. To increase in vivo stability of the CpG oligonucleotide, the oligonucleotide can be modified by the inclusion of one or more phosphorothioate modified nucleotides. Exemplary oligonucleotide sequences suitable for use in mouse (SEQ ID NO:1) and human (SEQ ID NOs:2-5).

Another aspect of the disclosure relates to methods of protecting neural cells (including hippocampal and cortical neurons) against excitotoxic brain injury. Such methods involve systemically administering to a subject an agent that binds to a Toll-like receptor (TLR) expressed on a cell of the periphery or in the central nervous system (CNS). The peripheral or CNS cells that express TLRs can be non-neural cells. For example, the non-neural cells can be immune cells, such as B cells, dendritic cells, macrophages or microglia. Agents that bind to various TLRs, including TLR2, TLR4, TLR7 and TLR9, among others, are useful in the methods disclosed herein. In one embodiment, the agent is an unmethylated CpG oligonucleotide that binds to and activates TLR9. In another embodiment, the agent is imiquimod or another agent that binds to and activates TLR7. In yet other embodiments, the agent is MALP-2, which binds to and activates TLR2 or a nontoxic analog LPS, which binds to and activates TLR4.

Excitotoxic brain injury can be the result of a variety of disparate events. For example, the disclosed methods are suitable for protecting cells from injury or death due to epilepsy, traumatic brain injury and Alzheimer's disease, as well as stroke. Following administration, the agent binds to a TLR and induces cellular changes (for example, in the genomic program), such as inducing production of one or more neuroprotective cytokines, such as TGFβ, TNFα, and IFNβ.

To exert a protective preconditioning effect, the agent that binds to the TLR is administered prior to the excitotoxic event. For example, the agent can be administered to a subject identified as being at risk for an excitotoxic brain injury. In one exemplary application, the agent is administered to a subject prior to a surgical procedure, such as a surgical procedure involving the CNS or cardiovascular system. For example, such methods can be employed to protect a subject from excitotoxic brain injury resulting from surgical procedures involving arterial bypass, which are associated with an increased risk of excitotoxic brain injury, such as endarterectomy, pulmonary bypass and coronary artery bypass surgeries. Surgical interventions are typically non-recurring events; thus, the agent can be administered prior to the event in a single dose delivered prior to the start of the event. For optimal preconditioning effects, the agent is usually administered at least about 10 hours prior to the event (for example, surgery), and can be administered up to about 1 week prior to the event. Optionally, more than one doses of the agent are administered prior to the event.

Another aspect of the disclosure relates to methods of protecting non-neural cells against ischemia by systemically administering an agent that binds to a Toll-like receptor (TLR). Typically, the TLR is expressed by a cell other than a cell of the central nervous system. For example, the non-neural cell can be a muscle cell (including a skeletal, smooth or cardiac muscle cell), a kidney cell a liver cell, an endothelial cell or a cell of the immune system (such as a macrophage or microglial cell). In certain cases, the ischemic event is associated with a surgical procedure, such as coronary artery bypass surgery. As discussed above, with respect to other preconditioning regimens, the agent is administered prior to the onset of ischemia. In an embodiment, the agent binds to TLR9. In another embodiment, the agent binds to TLR7 (and/or TLR8). In yet other embodiments, the agents bind to TLR2 or TLR4. Exemplary agents include CpG oligonucleotides, imiquimod, MALP-2 and nontoxic LPS analogs.

Additional technical details are provided under the specific topic headings below.

### Terms

Unless otherwise explained, all technical and scientific terms used herein have the same meaning as commonly understood by one of ordinary skill in the art to which this disclosure belongs. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

The singular terms “a,” “an,” and “the” include plural referents unless context clearly indicates otherwise. Similarly, the word “or” is intended to include “and” unless the context clearly indicates otherwise. The term “plurality” refers to two or more. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described herein. The term “comprises” means “includes.” The abbreviation, “e.g.” is derived from the Latin exempli gratia, and is used herein to indicate a non-limiting example. Thus, the abbreviation “e.g.” is synonymous with the term “for example.”

In order to facilitate review of the various embodiments of this disclosure, the following explanations of specific terms are provided:

The phrase “excitotoxic injury” or “excitotoxic brain injury” refers to injury (including death), of neural cells, particularly neural cells of the brain, due to excessive stimulation of cell-surface receptors. Most commonly, excitotoxic injury is mediated through glutamate receptors, for example, by overactivation of N-methyl-d-aspartate (NMDA)-type glutamate receptors, resulting in excessive Ca²⁺ influx through the receptor's associated ion channel. Excitotoxic injury is believed to play a role in diverse conditions, including epilepsy, traumatic injury, and Alzheimer's disease.

The term “hypoxia” refers to a lack of oxygen. In a physiological context, the term hypoxia refers to an insufficiency of oxygen at a cellular, tissue or organismal level. Hypoxia can be caused by, for example, the reduction in partial pressure of oxygen (in the blood or in a tissue), inadequate oxygen transport (for example, due to a failure of oxygenated blood to reach a target tissue or cell), or the inability of the tissues to use oxygen. The term “infarct” refers to cell or tissue death due to a localized lack of oxygen (hypoxia).

Frequently, hypoxia is the result of “ischemia,” the reduction in oxygenated blood flow to a target tissue or organ. An “ischemic event” is an event or occurrence that results in decreased blood flow to a cell, collection or group of cells, tissue, or organ. Ischemic events include vasoconstriction, thrombosis and embolism, resulting in reduced blood flow to a tissue or organ.

The term “stroke” refers to an interruption of the blood supply to any part of the brain. A stroke can be due to an ischemic event (for example, occlusion of a blood vessel due to a thrombus or an embolism) or hemorrhage (for example, of a cerebral blood vessel).

A subject is at “risk for a cytotoxic insult” or at “risk for an excitotoxic, ischemic or hypoxic event” if there is an increased probability that the subject will undergo a excitotoxic, ischemic or hypoxic event relative to the general population. Accordingly, risk is a statistical concept based on empirical and/or actuarial data. Commonly, risk can be correlated with one or more indicators, such as symptoms, signs, characteristics, properties, occurrences, events or undertakings, of a subject. For example, with respect to risk of stroke, indicators include but are not limited to high blood pressure (hypertension), atrial fibrillation, transient ischemic events, prior stroke, diabetes, high cholesterol, angina pectoris, and heart disease. More generally, risk indicators for hypoxic events include surgery, especially cardiovascular surgeries, such as endarterectomy, pulmonary bypass surgery or coronary artery bypass surgery. Additional risk factors or indicators include non-medical activities, such as motorcycle riding, contact sports and combat. Other risk factors are discussed herein, and yet more can be recognized by those of ordinary skill.

The term “protect” with respect to a excitotoxic or hypoxic event refers to the ability of composition or treatment regimen to prevent, reduce in severity, or otherwise lessen the effects of an excitotoxic or hypoxic event at a cellular, tissue or organismal level. Methods for measuring severity of effects of an excitotoxic or hypoxic event include neurological, including behavioral, indicia (e.g., ascertainable via neurological examination of a subject) as well as by evaluation of cellular and metabolic parameters, for example, by Computed Axial Tomography (CT scan, CAT scan); Magnetic Resonance Imaging (MRI scan, MR scan); Carotid Ultrasound, including Transcranial Doppler (TCD); Cerebral Angiography: (Cerebral arteriogram, Digital subtraction angiography [DSA]); Computed Tomographic Angiography: (CT-angiography, CT-A, CTA); Magnetic Resonance Angiography (MRA) and/or other diagnostic procedures known to those of ordinary skill in the art.

A “CpG oligonucleotide” or “CpG ODN” is a nucleotide molecule, typically between about 12 and 30 nucleotides in length and including at least one unmethylated cytosine-guanosine dinucleotide. Generally, the unmethylated CpG dinucleotide is located at the interior of the nucleotide sequence rather than at an end. Unmethylated CpG dinucleotides are found throughout various genomes, including those of many bacteria and viruses. However, in the context of this disclosure, the CpG oligonucleotide is a synthetic (or isolated) nucleotide sequence. In some cases, the CpG oligonucleotide includes one or more nucleotide with a phosphorothioate modified backbone to increase stability of the CpG oligonucleotide in vivo.

A “Toll-like receptor” or “TLR” is a type I transmembrane protein which acts as a pattern recognition receptor (PRR). Toll-like receptors have been shown to play a role in innate immunity, for example, by recognizing conserved microbial structures or Pathogen-Associated Molecular Patterns (PAMP). More than a dozen TLRs are known, and the nucleic acids that encode them have been described. For example, nucleic acid sequences that encode human TLRs can be found, e.g., in GENBANK®: Accession nos. U88540 (TLR1); U88878 (TLR2); U88879 (TLR3); U88880 (TLR4) AB060695 (TLR5); AB020807 (TLR6); AF245702 (TLR7); AF245703 (TLR8); AF245704, and splice variants AF259262 and AF259263 (TLR9); and AF296673 (TLR10), among others, the sequences of which are well known. Naturally occurring and artificial ligands of several TLRs have been characterized. For example, peptidoglycan fragments (glycopeptides) bind to TLR4; dsRNA (a viral product) binds to TLR3; LPS (a component of bacterial cell walls) binds to TLR4; bacterial flagellin binds to TLR5; single stranded RNA (such as viral RNA) binds to TLR7 and TLR8; and unmethylated CpG motifs (such as those found in the genome of bacteria and viruses) bind to TLR9.

A ligand is said to “activate” a receptor if the ligand binds to the receptor, and such binding results in the initiation of one or more signaling events, such as translocation or phosphorylation of the receptor and/or other signaling molecules.

The modifiers “systemic” and “systemically” are used in reference to administration/administering of a composition to indicate that administration results in the composition contacting cells and/or tissues at one or more sites at a distance to the site of administration, including cells and/or tissues of an organ or body part that is not the organ or body part into which the composition is directly administered. Most commonly, systemic administration involves introducing the composition directly or indirectly into the circulatory system of the organism. Thus, intravenous administration is one method of systemic administration of a composition. Additionally, a composition can be systemically administered by introducing the composition into a site that indirectly results in the composition being introduced into (either by diffusion or an active transport process) the circulatory system of the organism. Thus, intranasal, oral, transdermal, subcutaneous, intramuscular, intrathecal and intraperitoneal routes can all be systemic administration of the composition. The term systemic is used to distinguish the administration route from methods that result in a composition being retained in close proximity (for example, within the same tissue or organ) to the site of introduction.

A “subject” is a living multi-cellular vertebrate organism, a category that includes both human and veterinary subjects, including human and non-human mammals. In a clinical setting with respect to preconditioning against excitotoxic injury and/or hypoxia, a subject is usually a human subject, although veterinary subjects are also contemplated.

A “neural cell” is any cell in a lineage that originates with a neural stem cell and includes a mature neuron. Thus, the term neural cell includes neurons (nerve cells) as well as their progenitors regardless of their stage of differentiation. In the context of an adult brain, neural cells are predominantly differentiated neurons. In contrast, a “non-neural cell” is a cell of a lineage other than a neural cell lineage, that is a lineage that does not culminate in the differentiation of a mature neuron. The non-neural cell may reside in the central nervous system (CNS), for example, in the brain (such as glial cells and immune system cells, such as B cells, dendritic cells, macrophages and microglia), or may exist in an organ outside the CNS, such as cardiac, skeletal or smooth muscle (a muscle cell), liver (a hepatic cell) or kidney (a renal cell) and so forth. Non-neural cells include cells of the immune system, regardless of whether they reside in the CNS or elsewhere in the body of the organism.

A “cytoprotective cytokine” is a soluble protein (or glycoprotein) involved in the regulation of cellular proliferation and function that acts to preserve cellular function and prevent (or reduce) death of a cell in response to a stressful or otherwise aversive stimulus. Cytoprotective cytokines include transforming growth factor β (TGF-β, tumor necrosis factor α (TNFα), and type I interferons, such as interferon β (IFNβ. A “neuroprotective cytokine” is a cytoprotective cytokine that acts to preserve cellular function and reduce cell death in neural cells.

The term “medicament” is used interchangeably with the term “pharmaceutical composition.” Such compositions are formulated for administration to human and/or animal (veterinary) subjects, and typically include one or more active component (such as one or more of the CpG oligonucleotides disclosed herein) as well as one or more additional components to facilitate administration to a subject, for the therapeutic or prophylactic treatment (prevention or reduction) of a condition or disease. The additional components can include pharmaceutically acceptable carriers, buffers or excipients. Pharmaceutically acceptable carriers, buffers and so forth, are well known in the art, and are described, e.g., in Remingtons Pharmaceutical Sciences, 19^{th} Ed., Mack Publishing Company, Easton, Pa., 1995.

“Prophylactic” treatment refers to the treatment of a subject prior to the full manifestation of an event, condition or disease for the purpose of preventing or reducing the symptoms, signs or consequences of the event, condition or disease. Thus, in the context of the present disclosure, prophylactic treatment of an excitotoxic injury or hypoxia refers to the treatment of a subject prior to the occurrence of an excitotoxic or hypoxic event (that is, prior to a first excitotoxic or hypoxic event, or prior to a subsequent excitotoxic or hypoxic event, or prior to the completion or culmination of an ongoing or recurrent excitotoxic or hypoxic event) and prior to the completion of the natural consequences and/or sequelae of the event.

A “preconditioning dose” is a dose of an effective compound, or composition containing such a compound, that protects a cell against injury or death due to an excitotoxic, ischemic or hypoxic event. The dosage of the effective compound or composition varies from compound to compound and between species. A suitable preconditioning dose for any compound can be determined empirically.

### Preconditioning

Exposure of cells to subthreshold levels (that is, at a level below that which causes injury) of a stressful (e.g., cytotoxic) stimulus can induce tolerance to subsequent events that would otherwise result in injury. This effect has been termed preconditioning, and is relevant to preventing or reducing injury due to cytotoxic insult such as excitotoxic events and hypoxia (e.g., due to ischemia) in a variety of cell and tissue types, including neural cells, muscle cell (e.g., skeletal as well as cardiac muscle cells), kidney cells and liver cells.

Preconditioning in the brain (that is, of neural cells) and other organs can be produced following exposure to a subthreshold level of an otherwise toxic stimulus. For example, brief exposure to ischemia and administration of a sub-toxic dosage of lipopolysaccharide (LPS) have been shown to elicit a protective response to subsequent ischemic events. This effect is dependent on de novo protein synthesis, and involves changes in genomic programming associated with inflammation.

Following administration of a suitable preconditioning agent (such as a CpG oligonucleotide, imiquimod, or other agent that activates a TLR), protection against excitotoxic and/or hypoxic injury typically begins within about 10-12 hours and lasts for up to several weeks, or more. In addition, protection can be extended by repeated administration of the agent.

The activation of inflammatory pathways is involved in preconditioning against excitotoxic injury, ischemia and hypoxia. For example, TNFα and its downstream signaling mediator, ceramide, are involved in achieving a preconditioning effect, and blockade of TNFα (with a soluble TNF receptor or fragment thereof) prevents the protective effect of preconditioning. Thus, proximal members of the TNFα pathway, namely TNFα and its receptors, TNFR1 (p55) and TNFR2 (p75), as well as sphingomyelin-based second messengers such as ceramide, are likely mediators of the protective effects of TNFα in LPS preconditioning. TNFα-activation of NF-κB may also be involved, as inflammatory molecules regulated by NF-κB, such as superoxide dismutase (SOD), have been shown to be involved in preconditioning (Bordel et al., J. Cereb. Blood Flow Metab. 20:1190-1196, 2000).

Interferons are also involved in cytoprotection against excitotoxic injury, ischemia and hypoxia. IFNs are a family of cytokines comprised of type I (IFNα and IFNβ) and type II (IFNγ) IFNs. First characterized based on anti-viral properties, type I IFNs have many immunomodulatory functions. Generally, IFNα/β are associated with anti-inflammatory cytokines (Shnyra et al., J. Immunol. 160:3729-3736, 1998).

IFN has been shown to improve stroke outcome following systemic administration in animal models (Veldhuis et al., J. Cereb. Blood Flow Metab. 23:1029-1039, 2003; Liu et al., Neurosci Lett. 327:146-148, 2002). The mitigating role of IFNβ in stroke is primarily due to its anti-inflammatory properties that reduce cell infiltration into the affected tissue via regulation of matrix metalloproteinase-9. In addition, IFNβ has been shown to decrease reactive oxygen species, suppress inflammatory cytokines (Hua et al., J. Neurochem. 83:1120-1128, 2002) and promote cell survival (Barca et al., J. Neuroimmunol., 139:155-159, 2003), functions that contribute to improved outcome following stroke.

The basis for some of IFNα/β's regulatory functions lies in their action as a facilitator of expression of other IFN-inducible proteins known as IFN regulatory factors (IRFs), that in turn transactivate additional IFN-inducible genes (Taniguchi et al., Annu. Rev. Immunol. 19:623-655, 2001). IRFs constitute a family of transcription factors whose functions in some instances are distinct and independent of one another, while in others, appear to be interdependent (Taniguchi and Takaoka, Curr. Opin. Immunol. 14:111-116, 2002). For example, IRF3 binding to the interferon stimulated response element (ISRE) induces IFNβ which is involved in the early stages of preconditioning. The ability of IRF3 to transactivate IFNβ in this scenario depends on NFκB as well. Interaction between these two transcription factors is extensive; IRF3-NFκB complexes have been shown to interact not only at the ISRE but at κB sites as well (Wietek et al., J. Biol. Chem. 278:50923-50932, 2003; Leung et al., Cell 118:453-464, 2004). Furthermore, many genes contain both IRSE and κB sites within their promoter regions and depend upon interaction between the two factors for transcription initiation (Genin et al., J. Immunol. 164:5352-5361, 2000). IRF3 is induced by agents that activate TLRs and is likely to mediate the cytoprotective effects of preconditioning agents.

Preconditioning involves a fundamental change in the genomic program or response (that is, the pattern of gene expression produced in response) to excitotoxic, ischemic and/or hypoxic injury that shifts the outcome from cell death to cell survival (Stenzel-Poore et al., The Lancet 362:1028-1037, 2003). This change in gene expression, or genomic reprogramming, in response to cytotoxic insults, such as excitotoxic, ischemic and/or hypoxic events, involves a pronounced suppression of gene expression (for example, of inflammatory cytokines, and certain ion channels and channel regulators, e.g., K⁺ and Ca⁺⁺ channels, such as glutamate receptors), which is ordinarily injurious. Such suppression contrasts sharply with the upregulation of mRNA by excitotoxic, ischemic and/or hypoxic events without preconditioning. This change is not simply the lack of a response, but rather a reprogramming of the genomic response that involves the downregulation of genes that control metabolism, cell-cycle regulation, and, in neural cells, ion-channel activity. Additionally, in certain cells of the immune system, preconditioning elicits a shift from pro-inflammatory to anti-inflammatory cytokines.

Preconditioning in macrophages, leading to suppression of specific cytokines and inflammatory molecules, involves attenuation of NF-κB and AP-1 and enhanced expression of the signaling mediators, IRAK-M and SOCS-1 (Kobayashi et al., Cell 110:191-200, 2002; Nakagawa et al., Immunity 17:677-687, 2002; Kinjyo et al., Immunity 17:583-591, 2002). Similar genomic reprogramming is also likely to be involved in preconditioning in cardiac tissue (Meng et al., Am. J. Physiol. 275:C475-483, 1998), although the specific genes can differ (e.g., HSP70, c-jun, c-fos).

The present disclosure provides methods for preconditioning cells by systemically administering an agent that binds to a Toll-like receptor and thereby induces changes in the genomic program of certain cells, for example, as described above by altering the nature and amount of cytokines produced. For example, an agent that activates a TLR (e.g., a CpG oligonucleotide) can be administered to a subject using a systemic administration route. Binding of the agent to TLR expressed on the surface of target cells, for example, certain immune system cells, including B cells, dendritic cells (DC), macrophages, and microglial cells, results in genomic reprogramming and in the case of the above mentioned immune cells, can induce an alteration in the cytokine secretion profile, including the induction of cytoprotective cytokines, such as TNF-α, type I interferons (e.g., IFN-α IFN-β) and/or TGF-β.

### Selecting Subjects at Risk for Cytotoxic Insult

The methods disclosed herein are applicable to any cell types susceptible to excitotoxic, ischemic and/or hypoxic injury, which are amenable to preconditioning. For example, neural cells (including, e.g., hippocampal neurons and cortical neurons), muscle cells (including cardiac and striated muscle cells), hepatic cells and renal cells can be protected against injury and death by administering a preconditioning agent (such as a CpG oligonucleotide) prior to the occurrence of an excitotoxic, ischemic or hypoxic event. Thus, a preconditioning agent is typically administered to a subject that has been identified as having (e.g., diagnosed with) one or more risk factors indicative of an increased likelihood, relative to the general population or to a subject without the risk factor, of having an excitotoxic, ischemic and/or hypoxic event.

Excitotoxic injury results from excessive stimulation of cells (typically neural cells in the CNS) by certain neurotransmitter (e.g., glutamate) receptors. Excitotoxic injury can be a result of a condition that causes excessive chemical or electrical activity in the brain or it can be a result of conditions that cause a decrease in inhibitory or regulatory functions of the brain. Excitotoxic injury in the brain is associated with a variety of conditions with disparate etiologies and symptoms, including epilepsy, traumatic brain injury and Alzheimer's disease. For example, in addition to medical indications such as epilepsy or Alzheimer's disease, non-medical indicators of risk, pertaining to behaviors or activities that are statistically associated with an increased likelihood of injuries that can include an excitotoxic component. For example, traumatic brain injury (regardless of its cause) frequently involves an excitotoxic (and can also include) a hypoxic component. Thus, participation in activities that increase the risk of traumatic brain injury are indicators that can be used to select a subject for administration of a preconditioning agent (such as a CpG oligonucleotide). Such activities include, for example, motorcycle riding, motor vehicle racing, skiing, contact sports (such as, football, hockey, rugby, soccer, lacrosse, martial arts, boxing and wrestling), and the like. Additionally, impacts or wounds resulting from gunshot or explosives frequently cause traumatic brain injury. Accordingly, activities that are associated with an increased risk of gunshot wounds or injury caused by explosive devices (for example, in combat situations) are an indicator of risk that can be used to select a subject for treatment according to the methods disclosed herein.

Hypoxia is typically associated with ischemic events in the CNS or elsewhere in the cardiovasculature, (such as cerebrovascular ischemia, or stroke, myocardial ischemia due to narrowing or blockage of the vessels of the heart, iatrogenic ischemia, due to surgical procedures, and the like). In addition, hypoxia can occur in utero due to conditions such as inadequate placental function (for example, due to abrupio placentae), preeclamptic toxicity, prolapse of the umbilical cord, or complications from anesthetic administration. Additionally, injury by some hypoxic events (such as strokes) involves an excitotoxic component as well as a hypoxic component.

Thus, various cardiovascular signs and symptoms, such as atrial fibrillation, angina pectoris, hypertension, transient ischemic episodes and prior stroke, are all indicators of risk (or risk factors) that can be used to select a subject for administration of a preconditioning agent (such as a CpG oligonucleotide). Similarly, surgical procedures, especially those specifically involving the cardiovascular system, such as endarterectomy, pulmonary bypass and coronary artery bypass surgeries, are indicators of risk that can be used to select a subject for administration of a preconditioning agent.

### CpG Oligonucleotides

In one exemplary embodiment, oligonucleotides containing an unmethylated CpG motif are administered to a subject for the purpose of preconditioning one or more cells (or cell types, or tissues, or organs) to protect against a cytotoxic insult, such as an excitotoxic injury, ischemia or hypoxia.

In the context of the present disclosure, CpG oligonucleotides (or CpG ODN) are oligonucleotides that contain at least one unmethylated cytosine-guanine dinucleotide sequence. The production and use of CpG oligonucleotides are known in the art, and described, for example, in U.S. Pat. Nos. 6,194,388 and 6,406,705. Without being bound by theory, all compositions and methods of producing them disclosed in U.S. Pat. Nos. 6,194,388 and 6,406,705 are incorporated herein by reference for all purposes.

Typically, a CpG ODN is between about 8 and 100 nucleotides in length. The CpG ODN is at least 10, or at least 12 nucleotides in length. Generally, a CpG ODN is no more than about 40 nucleotides in length. Although longer nucleotides can be employed, the cost of production increases with length with no significant benefit in terms of activity. Thus, while it is possible to use CpG ODN longer than 50 nucleotides, or 60 nucleotides, or even 70, or 80, or 90, or 100 nucleotides or more in length, there is little benefit in doing so. Frequently, the unmethylated CpG is flanked by complementary nucleotides, such that a palindromic sequence capable of hairpin formation (via base pairing interactions) around the CpG dinucleotide is included in the sequence of the CpG ODN. The inclusion of palindromic sequences flanking the unmethylated CpG dinucleotide is particularly desirable when using short oligonucleotides (e.g., 10 or 12 nucleotides in length).

CpG oligonucleotides bind to Toll-like receptor 9 (TLR9) on the surface of cells in a wide variety of tissues and/or organs (Nishimura and Naito, Biol. Pharm. Bull. 28:886-892, 2005). Binding of a CpG oligonucleotide to TLR9 initiates a signaling pathway mediated by MyD88 and p38-MAPK, which induces expression of NF-κB and IFNβ, among other genomic changes.

CpG oligonucleotides can be divided into at least three classes based on their structural and functional attributes. For example, A-Class CpG oligonucleotides (exemplified by SEQ ID NO:2) stimulate dendritic cells to make large amounts of IFNα but have a weak effect on B cells. In contrast, B-Class CpG oligonucleotides (exemplified by SEQ ID NO:3 and SEQ ID NO:5) induce IFNα production to a lesser extent, but very strongly induce B cell activation and antibody production. C-Class oligonucleotides (exemplified by SEQ ID NO:4) combine the effects of A- and B-Class oligonucleotides by exhibiting strong B cell, IFNα stimulation, and natural killer cell activation. Any of these classes can be used to elicit a protective response in the context of the preconditioning methods disclosed herein.

For use in the methods disclosed herein, oligonucleotides can be synthesized de novo using any of a number of procedures well known in the art. For example, the β-cyanoethyl phosphoramidite method (Beaucage and Caruthers, Tet. Let. 22:1859, 1981); nucleoside H-phosphonate method (Garegg et al., Tet. Let. 27: 4051-4054, 1986; Froehler et al., Nucl. Acid. Res. 14: 5399-5407, 1986; Garegg et al., Tet. Let. 27: 4055-4058, 1986; Gafffney et al., Tet. Let. 29:2619-2622, 1988) can be used. These chemistries can be performed by a variety of automated oligonucleotide synthesizers available in the market. Alternatively, oligonucleotides can be prepared from existing nucleic acid sequences (e.g., genomic or cDNA) using known techniques, such as those employing restriction enzymes, exonucleases or endonucleases.

For use in vivo, it can be desirable to use an oligonucleotides that is relatively resistant to degradation (such as, by endo- and exo-nucleases). An oligonucleotide that is relatively resistant to in vivo degradation is referred to as a “stabilized oligonucleotide.” Oligonucleotide stabilization can be accomplished via phosphate backbone modifications. For example, an ODN can be rendered nuclease resistant by phosphorothioate modification (that is, at least one of the phosphate oxygens is replaced by sulfur) of one or more internucleotide linkages. In some cases, the terminal internucleotide linkages are phosphorothioate modified. Procedures for synthesizing phosphorothioate modified CpG oligonucleotides are disclosed, e.g., in U.S. Pat. Nos. 5,663,153 and 5,723,335, the disclosures of which are incorporated herein for all purposes.

The pharmacokinetics of phosphorothioate ODN show that they have a systemic half-life of forty-eight hours in rodents and are useful for in vivo applications (Agrawal et al. Proc. Natl. Acad. Sci. USA 88:7595-7599, 1991). Phosphorothioates may be synthesized using automated techniques employing either phosphoramidate or H phosphonate chemistries.

Other stabilized oligonucleotides include: nonionic DNA analogs, such as alkyl- and aryl-phosphonates (in which the charged phosphonate oxygen is replaced by an alkyl or aryl group), phosphodiester and alkylphosphotriesters, in which the charged oxygen moiety is alkylated. Oligonucleotides which contain a diol, such as tetraethyleneglycol or hexaethyleneglycol, at either or both termini have also been shown to be substantially resistant to nuclease degradation. Aryl- and alkyl-phosphonates can be made (for example, as described in U.S. Pat. No. 4,469,863); and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann and Peyman, Chem. Rev. 90:543-584, 1990; Goodchild, Bioconjugate Chem. 1:165-187, 1990).

For administration in vivo, CpG oligonucleotides can be associated with a molecule that enhances binding to target cell surfaces and/or increased cellular uptake by target cells to form an “oligonucleotide delivery complex.” Oligonucleotides can be ionically, or covalently associated with appropriate molecules using techniques which are well known in the art. A variety of coupling or crosslinking agents can be used, including protein A, carbodiimide, and N-succinimidyl-3-(2-pyridyldithio) propionate (SPDP). Alternatively, oligonucleotides can be encapsulated in liposomes or virosomes using well-known techniques to facilitate delivery.

Oligonucleotides containing at least one unmethylated CpG dinucleotide can be administered to a subject in vivo as a preconditioning agent to prevent or reduce the adverse effects of excitotoxic injury, ischemia and/or hypoxia. CpG oligonucleotides are systemically administered to a subject at risk of one or more of excitotoxic, ischemic and/or hypoxic injury prior to an event that is likely to cause such injury. For use as a preconditioning agent, an effective amount of an appropriate oligonucleotide (alone or formulated as an oligonucleotide delivery complex) can be administered to a subject by any mode allowing the oligonucleotide to bind to appropriate receptors on the surface of target cells (e.g., TLR9). Formulation and dosages of compositions containing CpG oligonucleotides are discussed in detail herein below.

Other Agents that Activate Toll-Like Receptors.

In addition to CpG oligonucleotides, other agents that bind to and activate TLRs can also be used as preconditioning agents to prevent or reduce the effects of excitotoxic injury and/or hypoxia. At least ten TLRs have been reported in humans (Janeway and Medzhitov, Annu. Rev. Immunol. 20:197-216, 2002) and nine in the mouse (Olson and Miller, J. Immunol. 173:3916-3924, 2004). TLRs are expressed on the surface of cells in a wide variety of tissues, including brain, heart, kidney, liver, lung, skeletal muscle, spleen and thymus. In addition overlapping subsets of TLRs are expressed on different cells of the immune system. For example, TLR9 is highly expressed on human dendritic cells and B cells, whereas TLR2 is most highly expressed on monocytes, as is TLR4. TLR1 is expressed well on monocytes, dendritic cells and B cells, as well as on NK cells and T cells. TLR 5 is also expressed on T cells, NK cells and monocytes, but little expression is seen on B cells or monocytes. TLR6 is expressed on all of the above cell lineages, with expression being highest on B cells. TLR7 is expressed on monocytes, B cells and dendritic cells, with highest expression in dendritic cells.

A common pathway in all the TLRs is the ability to induce NF□B, which subsequently leads to the transcription of various cytokines, chemokines and cell surface molecules (Andreakos et al., Immunol Rev. 202:250-265, 2004). This induction of NF-κB is involved in establishment of cytoprotection using preconditioning regimens. Thus, any agent that activates a Exemplary agents that stimulate TLRs (other than CpG oligonucleotides) and that are suitable for administration as preconditioning agents, include non-toxic analogs of LPS, which activate TLR4, and MALP-2 a TLR2 agonist, and imiquimod a TLR7/8 agonist.

For example, imiquimod (and other imidazoquinoline compounds, such as R-848) bind to and activate TLR7 in mice, and TLR7 and TLR8 in humans (Hemmi et al., Nat Immunol., 3:196-200, 2002; Jurk et al., Nat Immunol. 3:499, 2002). Previously, these compounds have been used as antiviral and antitumour agents, typically at doses ranging between 0.25 and 5 mg/kg. Preconditioning doses of imiquimod typically range from about 0.002 mg/kg (such as 0.005 mg/kg, or 0.008 mg/kg, or about 0.01 mg/kg, or about 0.02 mg/kg, or about 0.05 mg/kg) to about 0.1 mg/kg in humans, such as about 0.08 mg/kg). Administration of a preconditioning dose of imiquimod (and/or other imidazoquinoline compounds or derivative(s) that bind to and activate TLR7/8) to a subject at risk of an excitotoxic injury, ischemia and/or hypoxia, prior to such a cytotoxic insult protects against cell injury and death. Thus, imiquimod (and related compounds or derivatives) can be used as an alternative to, or in combination with CpG oligonucleotides as preconditioning agents.

Additional preconditioning agents can be identified using a reporter system in which binding and activation of a selected TLR and induction of NF-κB is detected using an NF-κB responsive reporter construct. Cell lines, such as HEK293, stably transfected with the components necessary for signaling via a selected TLR are transfected with an NFκB inducible reporter plasmid, pNiFty2-SEAP (InvivoGen, San Diego). This plasmid contains an engineered promoter that combines five NFκB sites with the proximal ELAM (endothelial cell-leukocyte adhesion molecule) promoter upstream of a reporter gene encoding secreted alkaline phosphatase (SEAP). SEAP is extremely heat stable and can be detected spectrophotometrically, either colorimetrically or by detecting a luminescent product, e.g., using a PHOSPHA-LIGHT™ chemiluminescence kit (Applied Biosystems, BP3000). In this assay, the substrate CSPD [3-(4-methoxyspiro[1,2-d]oxetane-3,2′(5′-chloro)-tricyclo[3.3.1.13,7)decane]-4-yl)phenyl phosphate] is dephosphorylated by SEAP, and the resulting unstable dioxetane anion decomposes and emits light at a wavelength of 477 nm. The light signal is quantitated in a microplate luminometer and is linear up to 5 orders of magnitude and proportional to the concentration of SEAP. The extent of TLR activation can be quantified by collecting supernatant and determining the concentration of SEAP via this assay.

Cell lines expressing mouse and human TLRs 1-10 are commercially available (e.g., from InvivoGen) or can be produced by those of skill in the art using routine molecular biology procedures, for example as described in Sambrook et al., Molecular Cloning: a Laboratory Manual, Cold Spring Harbor Press, 1989.

In brief, the transfected cell line expressing the selected TLR and the parental HEK293 cells each carrying the NF-κB reporter construct are stimulated (for between 12 and 24 hours, e.g., for approximately 18 hours or overnight) with varying doses of a test agent. Typically, each test agent is tested at multiple doses to determine a dose/response curve. Following the incubation, supernatant is collected and NF-κB activity is measured using an alkaline phosphatase assay.

Following detection of an agent that binds to and activates the selected TLR and induces NF-κB, the preconditioning capacity of the agent can be confirmed using an in vitro model of stroke. Primary mouse cortical neuronal cultures are pretreated with media or the identified test agent at a suitable dose determined from the dose/response curve. Approximately 24 hours later, the growth medium is replaced with PBS containing 0.5 mM CaCl₂ and 5 mM MgCl₂, pH 7.4. The neuronal cultures are placed in an anaerobic chamber (Form a Scientific) containing an atmosphere of 85% N₂, 5% H₂, 10% CO₂ that is maintained at 35° C. (oxygen-glucose deprivation-treatment, “OGD”). Following OGD-treatment (3 hours), the PBS is replaced with Minimum Essential Medium (MEM) and the cultures are returned to normoxic conditions.

Percent cell death is then determined. For example, background cell death can be determined using medium and the test agent alone without OGD, to determine the effect of each compound on cell viability. LPS (1 μg/ml) preconditioning of a TLR4 transfected cell line can be used as a positive control for neuronal protection. Cell death is assessed approximately 24 hours following OGD using, for example, the fluorescent exclusion dye propidium iodide. The percent cell death is quantified, and differences between means (% cell death) in the cells contacted with the test agent and controls are compared for significance, e.g., using between groups factorial ANOVA grouped on treatment (media vs. test agent) and hypoxic status (no OGD vs. OGD).

Agents that exhibit a significant protective effect, such as at least about a 20% decrease in cell death, as compared to cultures that were not exposed to the agent (e.g., medium alone), are suitable as preconditioning agents to protect against excitotoxic injury and/or hypoxia. The methods described above can be use to screen libraries of compounds, such as Mixture Based Positional Scanning Libraries, for preconditioning agents. A Mixture-Based Positional Scanning Library is designed to provide information on the activity of collections of systematically arranged compounds numbering in the thousands to millions. The positional scanning technology has been used successfully to identify novel enzyme inhibitors, receptor agonists and antagonists, antimicrobial, antifungal, and antiviral compounds (Houghten et al., J. Med. Chem. 42:3743-3778, 1999; Pinilla et al., Nat. Med. 9:118-122, 2003). In addition, this technology has been independently validated by a number of research groups. Publications from more than 100 separate studies carried out by approximately 50 research laboratories (Houghten et al., J. Med. Chem. 42:3743-3778, 1999) reflect the broad utility of screening systematically arranged collections of compounds, such as positional scanning libraries.

Each positional scanning library is designed around a core pharmacophore. Traditionally, core pharmacophores in positional scanning libraries are chosen based on the following criteria: the core structure can be produced under straightforward and inexpensive synthetic conditions; the core structure can have numerous incorporated functional diversity elements; and the core structure is known or purported to be of biological importance. Each positional scanning sub-library contains positions that enable structural variations around the central core. Screening data from a library provides extensive structure-activity relationship information and enables identification of active individual compounds. Thus the individual structural components and their representative contributions to total biological activity within the positional scanning library are revealed. Mixture-based small molecule positional scanning combinatorial libraries (Mixture Sciences, Inc.) can be screened to identify agents that activate a selected TLR. Typically, human TLR are utilized to identify agents with optimal activity characteristics for human receptors.

Thus, in an exemplary screening protocol, a transfected cell line expressing the selected TLR is first contacted with pools of libraries to identify libraries with active constituents. Library mixtures and library pools are formulated at 1 mg/ml in 10% dimethyl formamide (DMF). Cell lines are tested for toxicity to DMF at concentrations of 1% or less. Library pools are applied at the maximum practical concentration, determined by the cell line's tolerance to DMF.

Appropriate concentrations of DMF alone, as well as LPS in DMF controls, are run in duplicate in each 96 well assay plate. Following the incubation period, supernatant is collected for an alkaline phosphatase assay. Active libraries are screened subsequently as individual mixtures to identify the most active functional groups on each library scaffold. The compounds predicted to be most active are synthesized and tested as individual compounds. Ligands that show both TLR binding (and activation) and neuroprotective properties in the in vitro model can be further evaluated for their protective characteristics in an in vivo model of stroke.

### Pharmaceutical Compositions and Methods

The preconditioning compositions (medicaments) disclosed herein can be administered to a subject to protect against excitotoxic injury, ischemia and/or hypoxia. Accordingly, the compositions are administered to a subject at risk of an excitotoxic, ischemic or hypoxic event to prevent or reduce the deleterious effects of such an event or occurrence. Administration of the composition is not necessarily deemed to alter the likelihood of occurrence of any cytotoxic insult, rather administration of the preconditioning composition alters or modifies the outcome following the occurrence of such an event by inducing cellular changes (e.g., in the genomic program) that reduce, prevent or ameliorate the effects of the excitotoxic, ischemic and/or hypoxic event.

The preconditioning compositions (medicaments) include at least one agent that binds to a TLR. Thus, as disclosed herein, the pharmaceutical compositions include one or more agent that is a ligand of a TLR. The ligand is selected to be appropriate for the subject receiving the composition. For example, when administering a preconditioning agent to a human subject, the agent is selected to be a ligand that binds to and activates a human TLR. Similarly, if the subject to be treated is a non-human animal, the agent is selected to bind to and activate a TLR of that species of animal. It should be noted that some TLR ligands bind to human as well as animal TLRs, whereas other ligands bind TLRs of some but not other species. One of skill in the art can confirm appropriate TLR-binding of a selected ligand empirically without undue experimentation. In some cases, the composition includes a single TLR ligand; in other instances, the composition includes more than one TLR ligand. Where a composition includes more than one TLR ligand, the composition can include multiple agents that bind to and activate a single TLR (optionally with different signaling results) or that bind to and activate different TLRs.

The quantity of the TLR binding and activation agent (such as a CpG oligonucleotide or imiquimod) included in the pharmaceutical composition is an amount determined to provide a preconditioning effect. For example, when administered to a subject (such as a human subject) in one or more doses, a preconditioning composition can include an amount of a CpG oligonucleotide sufficient to provide at least about 0.005 mg of the CpG oligonucleotide per kg body weight of the subject (0.005 mg/kg). Thus, exemplary compositions include an amount of a CpG oligonucleotide from about 0.008 mg/kg (for example, about 0.01 mg/kg, or about 0.02 mg/kg, or about 0.025 mg/kg, or about 0.05 mg/kg) to about 0.2 mg/kg (for example, about 0.08 mg/kg, or about 0.09 mg/kg, or about 0.10 mg/kg, or about 0.12 mg/kg or about 0.15 mg/kg). Thus, for administration to an adult human, a the composition can be formulated to include at least about 0.1 mg (100 μg) of a CpG oligonucleotide, to about 100 mg of the CpG oligonucleotide, in a single dose. In another example, the TLR binding agent is imiquimod, which is administered at comparable doses (e.g., between about 0.005 and 0.2 mg/kg, such as between about 0.01 and 0.10, e.g., at approximately 0.05-0.08 mg/kg). Suitable dose ranges and dosage can be determined by one of skill in the art for any TLR binding agent with a preconditioning effect. Methods for formulating and delivering CpG oligonucleotides are provided in U.S. Pat. Nos. 6,194,388 and 6,406,705. The methods of formulating and administering CpG oligonucleotides disclosed therein are incorporated herein by reference.

The composition typically includes one or more pharmaceutically acceptable constituents, such as a pharmaceutically acceptable carrier and/or pharmaceutically acceptable diluent. Typically, preparation of a preconditioning composition (medicament) entails preparing a pharmaceutical composition that is essentially free of pyrogens, as well as any other impurities that could be harmful to humans or animals. Typically, the pharmaceutical composition contains appropriate salts and buffers to render the components of the composition stable and facilitate administration to a subject. Such components can be supplied in lyophilized form, or can be included in a diluent used for reconstitution of a lyophilized form into a liquid form suitable for administration. Alternatively, where the inactivated pathogen is prepared for administration in a solid state (e.g., as a powder or pellet), a suitable solid carrier is included in the formulation.

Aqueous compositions typically include an effective amount of the preconditioning agent dispersed (for example, dissolved or suspended) in a pharmaceutically acceptable diluent or aqueous medium. Pharmaceutically acceptable molecular entities and compositions generally do not produce an adverse, allergic or other undesirable reaction when administered to a human or animal subject. As used herein, pharmaceutically acceptable carriers include any and all solvents, dispersion media, coatings, isotonic and absorption delaying agents, and the like. Optionally, a pharmaceutically acceptable carrier or diluent can include an antibacterial, antifungal or other preservative. The use of such media and agents for pharmaceutically active substances is well known in the art. Except insofar as any conventional media or agent is incompatible with production of a preconditioning response, its use in the preconditioning compositions is contemplated. In some cases (for example, when liquid formulations are deemed desirable, or when the agent is reconstituted for multiple doses in a single receptacle), these preparations contain a preservative to prevent or inhibit the growth of microorganisms.

Pharmaceutically acceptable carriers, excipients and diluents are known to those of ordinary skill in the described, e.g., in Remington's Pharmaceutical Sciences, by E. W. Martin, Mack Publishing Co., Easton, Pa., 15th Edition (1975), describes compositions and formulations suitable for pharmaceutical delivery of inactivated pathogens.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

For example, the pharmaceutical compositions (medicaments) can include one or more of a stabilizing detergent, a micelle-forming agent, and an oil. Suitable stabilizing detergents, micelle-forming agents, and oils are detailed in U.S. Pat. Nos. 5,585,103; 5,709,860; 5,270,202; and 5,695,770. A stabilizing detergent is any detergent that allows the components of the emulsion to remain as a stable emulsion. Such detergents include polysorbate, 80 (TWEEN) (Sorbitan-mono-9-octadecenoate-poly(oxy-1,2-ethanediyl; manufactured by ICI Americas, Wilmington, Del.), TWEEN 40™, TWEEN 20™, TWEEN 60™, Zwittergent™ 3-12, TEEPOL HB7™, and SPAN 85™. These detergents are typically provided in an amount of approximately 0.05 to 0.5%, such as at about 0.2%.

A micelle forming agent is an agent which is able to stabilize the emulsion formed with the other components such that a micelle-like structure is formed. Such agents generally cause some irritation at the site of injection in order to recruit macrophages to enhance the cellular response. Examples of such agents include polymer surfactants described by, e.g., Schmolka, J. Am. Oil. Chem. Soc. 54:110, 1977, and Hunter et al., J. Immunol 129:1244, 1981, and such agents as PLURONIC™ L62LF, L101, and L64, PEG1000, and TETRONIC™ 1501, 150R1, 701, 901, 1301, and 130R1. The chemical structures of such agents are well known in the art. In one embodiment, the agent is chosen to have a hydrophile-lipophile balance (HLB) of between 0 and 2, as defined by Hunter and Bennett (J. Immun. 133:3167, 1984). The agent can be provided in an effective amount, for example between 0.5 and 10%, or in an amount between 1.25 and 5%.

The oil included in the composition is chosen to promote the retention of the pathogen in oil-in-water emulsion, and preferably has a melting temperature of less than 65° C., such that emulsion is formed either at room temperature, or once the temperature of the emulsion is adjusted to room temperature. Examples of such oils include squalene, squalane, EICOSANE™, tetratetracontane, glycerol, and peanut oil or other vegetable oils. In one specific, non-limiting example, the oil is provided in an amount between 1 and 10%, or between 2.5 and 5%. The oil should be both biodegradable and biocompatible so that the subject can break down the oil over time, and so that no adverse affects, such as granulomas, are evident upon use of the oil.

The pharmaceutical compositions (medicaments) can be prepared for use in preconditioning or prophylactic regimens and administered to human or non-human subjects to elicit a protective response against an excitotoxic, ischemic or hypoxic event. For example, the compositions described herein can be administered to a human (or non-human) subject to elicit a protective response against stroke or other ischemic events.

A pharmaceutical composition (for example, containing a CpG oligonucleotide) can be administered by any means known to one of skill in the art, such as by nasal, intravenous, intramuscular, or subcutaneous injection, but even oral, and transdermal routes are contemplated, so long as the route of administration results in systemic (as opposed to localized) distribution of the preconditioning agent. In one embodiment, administration is intranasal.

As an alternative to liquid formulations, the preconditioning composition can be administered in solid form, e.g., as a powder, pellet or tablet. For example, the preconditioning agent can be administered as a powder using a transdermal needleless injection device, such as the helium-powered POWDERJECT® injection device. This apparatus uses pressurized helium gas to propel a powder formulation of a preconditioning composition, e.g., containing a CpG oligonucleotide, at high speed so that the particles perforate the stratum corneum and contact cells in the epidermis.

Polymers can be also used for controlled release. Various degradable and nondegradable polymeric matrices for use in controlled drug delivery are known in the art (Langer, Accounts Chem. Res. 26:537, 1993). For example, the block copolymer, polaxamer 407 exists as a viscous yet mobile liquid at low temperatures but forms a semisolid gel at body temperature (Johnston et al., Pharm. Res. 9:425, 1992; and Pec, J. Parent. Sci. Tech. 44(2):58, 1990). Alternatively, hydroxyapatite has been used as a microcarrier for controlled release (Ijntema et al., Int. J. Pharm. 112:215, 1994). In yet another aspect, liposomes are used for controlled release as well as drug targeting of the lipid-capsulated drug (Betageri et al., Liposome Drug Delivery Systems, Technomic Publishing Co., Inc., Lancaster, Pa., 1993). Numerous additional systems for controlled delivery of therapeutic compositions are known (e.g., U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342; and 5,534,496).

Typically, but not always, the preconditioning compositions are administered prior to the occurrence of a excitotoxic, ischemic or hypoxic event (or prior to an increase in the likelihood of such an event). Generally, the composition is administered at least 10 hours prior to the event or activity, in order to fully realize the preconditioning effect of administration. Usually, the composition is administered at least 24 hours before the event or activity. The protective effects of a single administration of a preconditioning agent, such as a CpG oligonucleotide, last for greater than one week (e.g., up to about 10 days, or more). Thus, in the case of an isolated event, that is, an event that is not predicted to be a recurring event, such as a surgical operation, the composition is given prior to the commencement of the event, such as about 10 hours, or about 12 hours, or about 24 hours prior to the event or activity, and can be given up to about 1 week prior to the event, and in some cases up to about 10 days or more prior to the event. In the case of a recurrent event, such as repeated engagement in a contact sport, multiple administrations are given, the ultimate dose (that is, the most recent dose prior to the event) being given prior (such as, at least 10 hours, or up to about 1 week, prior) to the event or activity. Similarly, in the case of an ongoing event, such as in the case of Alzheimer's disease, multiple administrations are given, for example on a predetermined schedule, such as at weekly intervals. Alternatively, the composition can be formulated and administered on a continuous basis, for example using a pump (or other intravenous or intrathecal) infusion method. The individual treatment regimen can be customized to the particular event or activity, such that the protective effects of the preconditioning dose of the agent (such as a CpG oligonucleotide) are optimized under the particular circumstances for the particular subject.

It will be apparent that the precise details of the methods or compositions described can be varied or modified without departing from the spirit of the described invention. The following examples are provided to illustrate certain particular features and/or embodiments. These examples should not be construed to limit the invention to the particular features or embodiments described. Each of the references cited below is incorporated by reference for all purposes.

### EXAMPLES

### Example 1

### Preconditioning with CpG Oligonucleotide Confers Neuroprotection in an In Vitro Ischemia Model

This example provides an exemplary in vitro model of neuronal ischemia, and demonstrates that preconditioning with CpG oligonucleotides protects against hypoxia.

In vitro mouse neuronal cultures: Cortical neuronal cultures were prepared as described Jin et al., Neruochem. Res. 27:1105-1112, 2002) from E-16 mouse pups (C57Bl/6, Jackson labs). In brief, cortices were dissected and separated from meninges, olfactory bulbs, basal ganglia and hippocampi, and the cortices digested in 0.05% trypsin-EDTA for 15 min at 37° C. Cells were triturated and single cell suspension was plated at density of 5×10⁵ cells/ml. Cells were cultured in Neurobasal A medium (Invitrogen, Carlsbad) containing 2% B27, 2 mM Glutamate. Neuronal enrichment was determined by staining for neurons, microglia and astrocytes with cell specific markers.

In vitro neuronal ischemia model: Neuronal cultures were treated with varying doses of an exemplary CpG oligonucleotide (SEQ ID NO:1) in Neurobasal-A media supplemented with 1% Glutamax 24 hours prior to 3 hours oxygen-glucose-deprivation (OGD) treatment. OGD was performed by replacing medium with PBS containing 0.5 mM CaCl₂ and 5 mM MgCl₂, pH 7.4, and then placing the neuronal cultures in an anaerobic chamber (Form a Scientific) containing an atmosphere of 85% N₂, 5% H₂, 10% CO₂ maintained at 35° C. Following OGD-treatment (3 hours), PBS was replaced with Minimum Essential Medium (MEM) and the cells returned to normoxic conditions. Percent neuronal cell death was determined by propidium iodide staining in two different fields of view in duplicate, compared to total DAPI staining in identical fields.

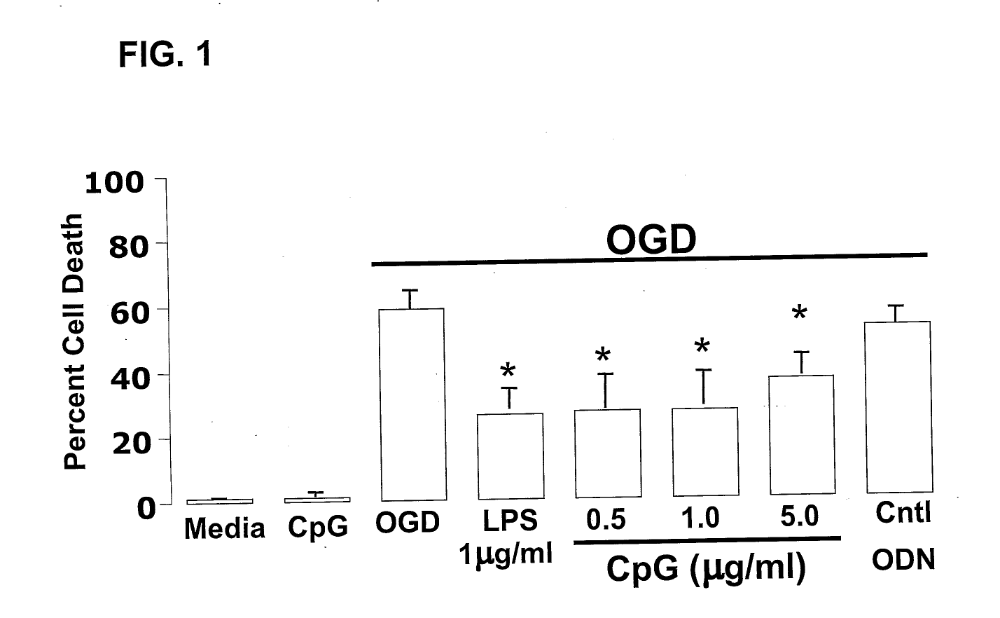

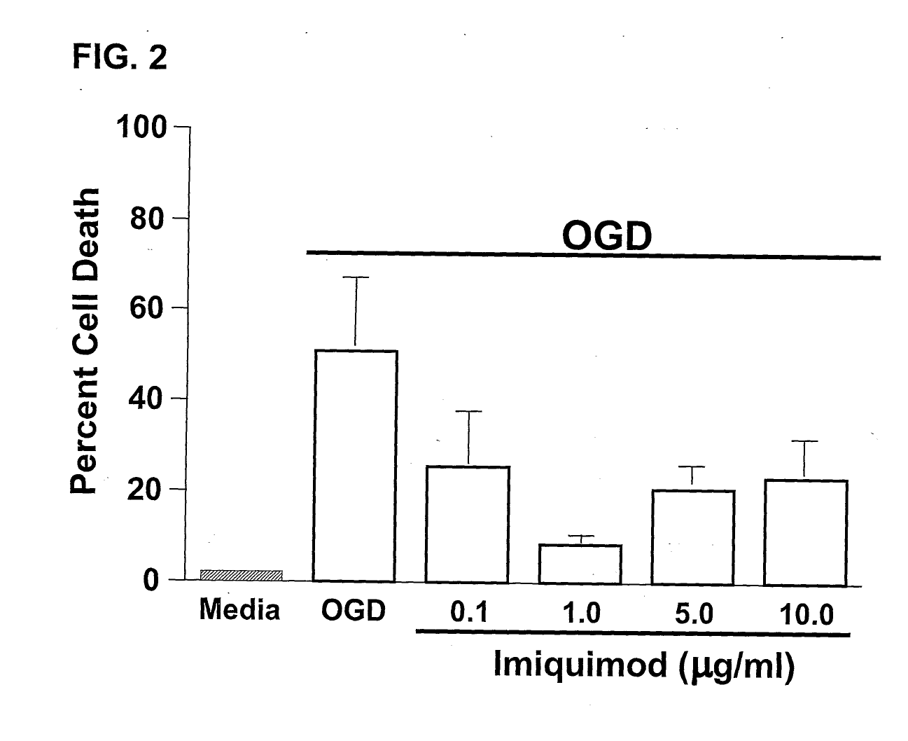

As shown in FIG. 1, the exemplary oligodeoxynucleotide containing an unmethylated CpG motif (SEQ ID NO:1), confers neuroprotection against oxygen-glucose deprivation in mouse neuronal cultures. Similar results were obtained with imiquimod, as shown in FIG. 2.

### Example 2

### NF-κB Induction by CpG Oligonucleotides in a TLR9 Expressing Cell Line

This example provides an exemplary reporter system for detecting binding and activation of a Toll-like receptor. Using this model, results are provided that demonstrate that CpG oligonucleotides that bind to TLR9 activate signaling via the receptor and induce NF-κB activity.

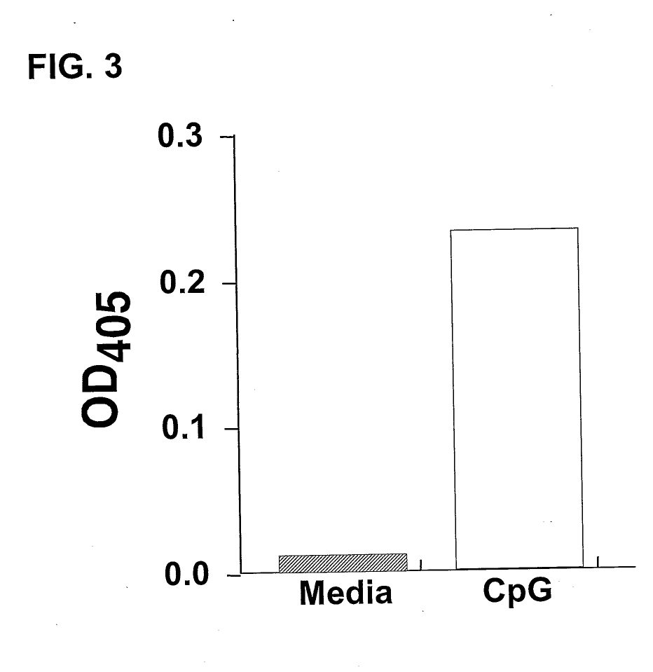

Human embryonic kidney cell line HEK293 was transfected with an expressible nucleic acid encoding human TLR9 and with an NFκB reporter construct (InvivoGen). The dual transfected cells were incubated with a 5 μM CpG oligonucleotide (SEQ ID NO:1) for 18 hours. Following stimulation with the CpG oligonucleotide (SEQ ID NO:1), the NFκB inducible reporter plasmid (pNiFty2-SEAP; InvivoGen) produced alkaline phosphatase, which was measured calorimetrically following substrate hydrolysis (FIG. 3).

### Example 3

### Preconditioning with an Exemplary CpG Oligonucleotide in an In Vivo Ischemic/Reperfusion Model

This example demonstrates that prophylactic administration of a composition containing a CpG oligonucleotide is neuroprotective in a mouse model of stroke.

Intraperitoneal Delivery. Preconditioning agent (20 μg CpG oligonucleotide (SEQ ID NO:1) in artificial cerebrospinal fluid (aCSF) or aCSF alone (control) was administered intraperitoneally to subject mice at designated timepoints prior to middle cerebral artery occlusion (MCAO) as described below.

Ischemic/Reperfusion Model. Following administration of a preconditioning agent or control composition, adult (˜3 months old) male C57BL/6 mice were subjected to 45 min MCAO according to the monofilament suture method previously described in detail (Hill et al., Brain Res. 820:45-54, 1999). Mice were anesthetized by halothane inhalation (4%/L O₂) and maintained with 1.5%/L O₂. The middle cerebral artery was blocked by a silicone-coated 8-0 monofilament nylon surgical suture that was threaded through the external carotid to the internal carotid and finally blocks the bifurcation into the MCA and anterior cerebral artery. The filament was maintained intraluminally for 45 min and then removed, thereby restoring blood flow. Cerebral blood flow (CBF) was monitored throughout the surgery by laser Doppler flowmetry (Periflow 5000; Perimed, Sweden). During and 2 hours following surgery, body temperatures was kept constant at 37° C. with a heating pad controlled by a thermostat. Body weights were monitored prior to and following MCAO. Neurological testing is performed prior to sacrifice as published previously (Hill et al., Brain Res. 820:45-54, 1999).

Motor Functions Tests. Following pretreatment and/or ischemia/reperfusion, damage due to stroke is assessed using several behavioral indices of neurological function. The corner test correlates with infarct volume and reveals post-infarct recovery (Wang et al., Stroke 35:1732-1737, 2004). The test measures the extent to which the mouse favors (turns toward) the ipsilateral side after moving into a confining corner. The assessment is conducted as previously described (Zhang et al., J. Neurosci Methods 117:207-214, 2002). Each mouse is tested 10 times per session. The footfault test, which assesses forelimb dysfunction, does not predict infarct size but reflects recovery after MCAO (Wang et al., Stroke 35:1732-1737, 2004) and neuroprotection (Gibson and Murphy, J. Cereb. Blood Flow Metab. 24:805-813, 2004). Mice are assessed for missteps while walking on an elevated wire grid. Data (footfaults) are expressed as a fraction of the total number of steps taken (Zhang et al., J. Neurosci Methods 117:207-214, 2002). The tactile adhesive removal test, which probes somatosensory function, is conducted as described (Lindner et al., J. Neurosci. 23:10913-10922, 2003). Briefly, small adhesive paper spots are attached to the distal portion of each forelimb and the time required to remove the paper with the mouth is determined (3 trials per sessions separated by 1 minute each). Additionally, mice are evaluated for neurological symptoms using other standard indicia of mouse behavior.

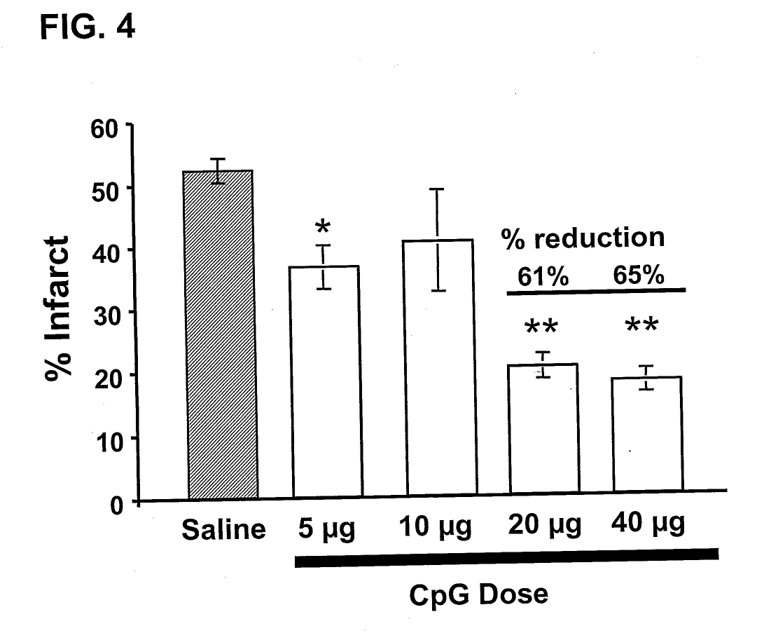

Infarct calculations. Following MCAO, mice were anesthetized with isoflurane and perfused with heparinized buffer to remove cells in the blood (Ford et al., J. Immunol. 154:4309-4321, 1995). Perfused brains were placed on a tissue slicer and sectioned into 1 mm thick coronal slices. To visualize the region of infarction, sections were stained with 1.5%, 2,3,4, triphenyltetrazolium chloride (TTC) in 0.9% phosphate buffered saline (Bederson et al., Stroke 17:1304-1308, 1986). Infarct size determination was performed using a computerized image analysis system according to principles described previously to eliminate edema measurement artifacts (Swanson et al., J. Cereb. Blood Flow Metab. 10:290-293, 1990). As shown graphically in FIG. 4, percent infarct was significantly decreased in mice treated with a preconditioning dose of an exemplary CpG oligonucleotide.

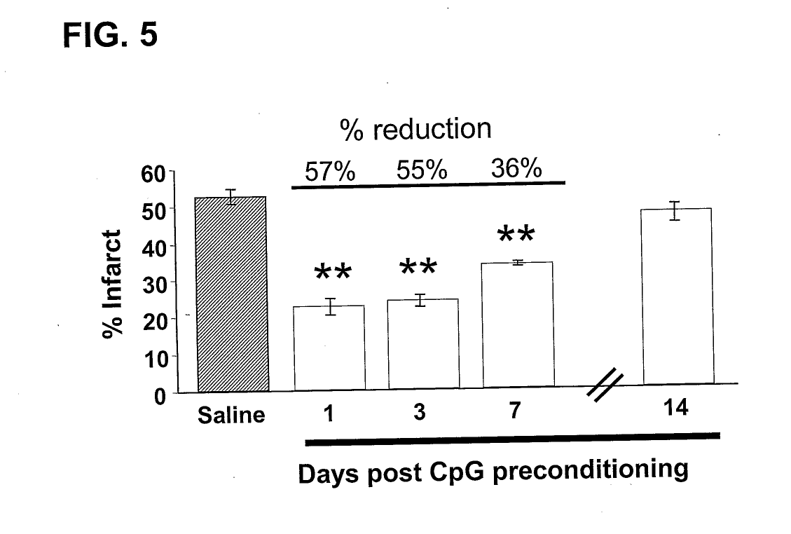

Time course of preconditioning. A time course for the preconditioning effects of CpG oligonucleotide administration was determined by administering 20 μg CpG oligonucleotide at intervals prior to MCAO, and evaluating percent infarct after MCAO as described above. Although peak preconditioning was observed following administration between 72 and 24 hours prior to MCAO, significant preconditioning was observed when a preconditioning dose of CpG oligonucleotide was administered up to a week prior to experimentally induced ischemia (FIG. 5).

### Example 4

### Preconditioning with Imiquimod in an In Vivo Ischemic/Reperfusion Model

This example demonstrates that prophylactic administration of imiquimod, a TLR7/8 binding agent, is neuroprotective in a mouse model of stroke.

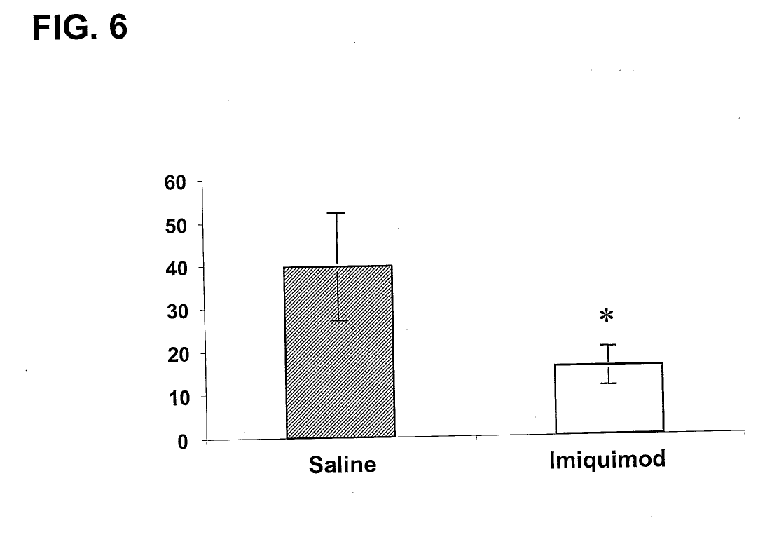

Imiquimod (20 μg) in artificial cerebrospinal fluid (aCSF) or aCSF alone (control) was administered intraperitoneally to subject mice 72 hours prior to 40 minute MCAO performed as described above. Brains were analyzed for infarct size as indicated in Example 3. FIG. 6 graphically illustrates that preconditioning with imiquimod protects against cell death in this in vivo model of stroke.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method of protecting a cell in a subject against excitotoxic injury, ischemia and/or hypoxia, the method comprising systemically administering to the subject a composition comprising a CpG oligonucleotide or imiquimod, thereby protecting the cell against excitotoxic injury or hypoxia.

2. The method of claim 1, comprising selecting a subject at risk for an excitotoxic, ischemic and/or hypoxic event.

3. The method of claim 2, wherein the risk is indicated by atrial fibrillation, one or more of transient ischemic events, a stroke, hypertension, and/or a surgical procedure.

4. (canceled)

5. The method of claim 3, wherein the surgical procedure is a vascular surgical procedure.

6. (canceled)

7. The method of claim 1, comprising preconditioning the cell by administering the composition comprising the CpG oligonucleotide prior to an excitotoxic, ischemic and/or hypoxic event.

8. The method of claim 7, comprising administering the composition comprising the CpG oligonucleotide at least about 10 hours prior to the excitotoxic, ischemic and/or hypoxic event.

9. The method of claim 7, comprising administering a plurality of doses of the composition comprising the CpG oligonucleotide, wherein the ultimate dose is administered within 1 week prior to the excitotoxic, ischemic and/or hypoxic event.

10. The method of claim 1, wherein the cell is a neural cell, a muscle cell, a liver cell, a kidney cell, an endothelial cell or an immune system cell.

11-12. (canceled)

13. The method of claim 1, wherein the subject is human.

14. The method of claim 1, wherein the hypoxia is associated with hypoxia in utero or an ischemic event.

15. (canceled)

16. The method of claim 1, wherein the excitotoxic injury is associated with epilepsy or traumatic brain injury.

17-21. (canceled)

22. The method of claim 1, comprising administering the composition comprising the CpG oligonucleotide to a subject intranasally, transdermally, orally, intrathecally, intravenously or intraperitoneally.

23. (canceled)

24. The method of claim 1, wherein the CpG oligonucleotide activates a Toll-like receptor 9 (TLR9).

25. The method of claim 1, wherein the CpG oligonucleotide comprises the sequence:
5′-tccatgacgttcctgacgtt-3′ (SEQ ID NO:1);
5′-gggggacgatcgtcgggggg-3′ (SEQ ID NO:2);
5′-tcgtcgttttgtcgttttgtcgtt-3′ (SEQ ID NO:3);
5′-tcgtcgtcgttcgaacgacgttgat-3′ (SEQ ID NO:4); or
5′-tgactgtgaacgttcgagatga-3′ (SEQ ID NO:5).

26. (canceled)

27. The method of claim 1, wherein the CpG oligonucleotide comprises at least one phosphorothioate modified nucleotide.

28. The method of claim 1, comprising administering a preconditioning dose of the CpG oligonucleotide.

29. The method of claim 28, comprising administering a preconditioning dose of the CpG oligonucleotide of at least about 0.005 mg/kg and no more than about 0.5 mg/kg.

30. The method of claim 28, comprising administering a preconditioning dose of the CpG oligonucleotide of at least about 0.02 mg/kg and no more than about 0.2 mg/kg.

31. (canceled)

32. A method of protecting a neural cell against excitotoxic brain injury, the method comprising: systemically administering to a subject an agent that binds to and activates a Toll-like receptor, which Toll-like receptor is expressed by at least one cell of the central nervous system or the periphery, thereby protecting the neural cell against excitotoxic brain injury.

33. The method of claim 32, comprising selecting a subject at risk for an excitotoxic event.

34. (canceled)

35. The method of claim 32, wherein the excitotoxic brain injury is associated with epilepsy, traumatic brain injury or Alzheimer's disease

36. (canceled)

37. The method of claim 32, comprising administering the agent prior to an excitotoxic event.

38. The method of claim 32, comprising selecting a subject at risk for an excitotoxic event.

39. The method of claim 32, wherein the agent that binds to and activates a Toll-like receptor is a CpG oligonucleotide that binds to and activates TLR9 or wherein the agent that binds to and activates a Toll-like receptor is imiquimod, which binds to and activates TLR7 and/or TLR8.

40. (canceled)

41. A method of protecting a non-neural cell against ischemia, the method comprising: systemically administering to a subject an agent that binds to a Toll-like receptor expressed by at least one cell of a tissue other than the central nervous system.

42. The method of claim 41, comprising selecting a subject at risk of ischemia.

43-45. (canceled)

46. The method of claim 41, wherein the ischemia is associated with a surgical procedure.

47-48. (canceled)

49. The method of claim 41, comprising administering the agent prior to an ischemic event.

50. The method of claim 41, wherein the agent that binds to and activates a Toll-like receptor is a CpG oligonucleotide that binds to and activates TLR9.

51. The method of claim 41, wherein the agent that binds to and activates a Toll-like receptor is imiquimod, which binds to and activates TLR7 and/or TLR8.

52. The method of claim 14, wherein the hypoxia is associated with an ischemic event.

53. The method of claim 52, wherein the ischemic event comprises cerebrovascular ischemia.
